(19) United States
(12) Patent Application Publication
(10) Pub. No.: US 2024/0074817 A1
(43) Pub. Date: Mar. 7, 2024

(54) SURGICAL PERCEPTION FRAMEWORK FOR ROBOTIC TISSUE MANIPULATION

(71) Applicant: THE REGENTS OF THE UNIVERSITY OF CALIFORNIA, Oakland, CA (US)

(72) Inventors: Florian RICHTER, La Jolla, CA (US); Michael YIP, La Jolla, CA (US); Yang LI, La Jolla, CA (US)

(21) Appl. No.: 18/273,819

(22) PCT Filed: Feb. 3, 2022
(86) PCT No.: PCT/US2022/015139

§ 371 Date: Jul. 24, 2023

Related U.S. Application Data
(60) Provisional application No. 63/145,100, filed on Feb. 3, 2021.

(51) Int. Cl. A61B 34/20; A61B 1/00; A61B 34/10; A61B 34/30; A61B 90/00; G06T 7/13; G06T 7/593; G06T 7/73; G06T 17/20

(52) U.S. Cl. CPC A61B 34/20; A61B 1/00193; A61B 34/10; A61B 34/30; A61B 90/37; G06T 7/13; G06T 7/593; G06T 7/73; G06T 17/205; A61B 2034/105; A61B 2034/2057; A61B 2034/2065; A61B 2090/365; G06T 2207/10068; G06T 2207/20081; G06T 2207/30004; G06T 2207/30204; G06T 2210/41

(57) ABSTRACT

A method for tracking a surgical robotic tool being viewed by an endoscopic camera, images of the surgical tool are received from the endoscopic camera and surgical tool joint angle measurements are received from the surgical tool. Predetermined features of the surgical tool on the images of the surgical tool are detected to define an observation model to be employed by a Bayesian Filter. A lumped error transform and observable joint angle measurement errors are estimated using the Bayesian Filter. The lumped error transform compensates for errors in a base-to-camera transform and non-observable joint angle measurement errors. Pose information over time of the surgical tool is determined with respect to the endoscopic camera using kinematic information of the robotic tool, the surgical tool joint angle measurements, the lumped error transform and the observable joint angle measurement errors. The pose information is provided to a surgical application.

### BACKGROUND

Surgical robotic systems, such as the da Vinci robotic platform (Intuitive Surgical, Sunnyvale, CA, USA), are becoming increasingly utilized in operating rooms around the world. Use of the da Vinci robot has been shown to improve accuracy through reducing tremors and provides wristed instrumentation for precise manipulation of delicate tissue. Current research has been conducted to develop new control algorithms for surgical task automation. Surgical task automation could reduce surgeon fatigue and improve procedural consistency through the completion of tasks such as suturing and maintenance of hemostasis.

Significant advances have been made in surgical robotic control and task automation. However, the integration of perception into these controllers is deficient. Perception for control tasks requires tracking the environment in 3D space. Tracking in this instance is defined as knowing the object of interest's location through time (e.g., a specific location on the tissue while being stretched). Without properly integrating perception, control algorithms will never be successful in non-structured environments, such as those under surgical conditions.

### SUMMARY

In one aspect, systems and methods are described herein for tracking a surgical robotic tool being viewed by an endoscopic camera. The method includes: receiving images of the surgical robotic tool from the endoscopic camera; receiving surgical robotic tool joint angle measurements from the surgical robotic tool; detecting predetermined features of the surgical robotic tool on the images of the surgical robotic tool to define an observation model to be employed by a Bayesian Filter; estimating a lumped error transform and observable joint angle measurement errors using the Bayesian Filter, the lumped error transform compensating for errors in a base-to-camera transform and non-observable joint angle measurement errors; determining pose information over time of the robotic tool with respect to the endoscopic camera using kinematic information of the surgical robotic tool, the surgical robotic tool joint angle measurements, the lumped error transform estimated by the Bayesian Filter and the observable joint angle measurement errors estimated by the Bayesian Filter; and providing the pose information to a surgical application for use therein.

In accordance with one particular implementation, the surgical application is a closed loop control system for controlling the robotic tool in a frame of view of the endoscopic camera.

In accordance with another particular implementation, the surgical application is configured to render the surgical robotic tool using the pose information.

In accordance with another particular implementation, the surgical robotic tool is rendered for use in an artificial reality or virtual reality system.

In accordance with another particular implementation, the surgical robotic tool and the endoscopic camera are located at a surgical site.

In accordance with another particular implementation, the endoscopic camera is incorporated in an endoscope incorporated in a robotic system that includes the surgical robotic tool.

In accordance with another particular implementation, the endoscopic camera is incorporated in an endoscope that is independent of a robotic system that includes the surgical robotic tool.

In accordance with another particular implementation, the surgical robotic tool joint angle measurements are received from encoders associated with the surgical robotic tool.

In accordance with another particular implementation, detecting predetermined features of the surgical robotic tool includes detecting point features.

In accordance with another particular implementation, detecting the point features is performed using a deep learning technique or fiducial markers.

In accordance with another particular implementation, the predetermined features are edge features.

In accordance with another particular implementation, detecting the edge features is performed using a deep learning algorithm or a canny edge detection operator.

In accordance with another aspect of the systems and methods described herein, a method for tracking tissue being viewed by an endoscopic camera includes: receiving images of the tissue from the endoscopic camera; estimating depth from the endoscopic images; initializing a three-dimensional (3D) model of the tissue with surfels from an initial one of the images and the depth data of the tissue to provide a 3D surfel model; initializing embedded deformation (ED) nodes from the surfels, wherein the ED nodes apply deformations to the surfels to mirror actual tissue deformation; generating a cost function representing a loss between the images from the endoscopic camera and the depth data of the tissue and the 3D surfel model; updating the ED nodes by minimizing the cost function to track deformations of the tissue; updating the surfels from the ED nodes to apply the tracked deformations of the tissue on the surfels; and adding surfels to grow a size of the 3D Surfel model based on additional information of the actual tissue that is subsequently captured in the images and the depth data to provide an updated 3D surfel model for use in a surgical application.

In accordance with one particular implementation, adding surfels further comprises adding, deleting and/or fusing the surfels to refine and prune the 3D surfel model and grow a size of the 3D surfel model based on additional information of the actual tissue that is subsequently captured in the images and the depth data.

In accordance with another particular implementation, the cost function is minimized by an optimization technique selected from the group including gradient descent, a Levenberg Marquardt algorithm and coordinate descent.

In accordance with another particular implementation, estimating depth from endoscopic images is performed using a stereo-endoscope and pixel matching or by directly estimating depth from a mono endoscope using a deep learning technique.

In accordance with another particular implementation, the method further includes removing irrelevant data from the images and the depth data.

In accordance with another particular implementation, the irrelevant data includes image pixels of a surgical tool.

In accordance with another particular implementation, the cost function includes a normal-difference cost.

In accordance with another particular implementation, the cost function includes a rigid-as-possible cost.

In accordance with another particular implementation, the cost function includes a rotational normalizing cost to constrain a rotational component of the ED nodes to the rotational manifolds.

In accordance with another particular implementation, the cost function includes a texture loss between matched feature points though matched feature point pairs.

In accordance with another particular implementation, the surgical application is a closed loop control system for controlling a robotic tool in a frame of view of the endoscopic camera.

In accordance with another particular implementation, the surgical application is configured to render the tissue using the updated 3D surfel model.

In accordance with yet another aspect of the systems and methods described herein, a method for synthesizing surgical robotic tool pose information and a deformable 3D reconstruction of tissue into a common coordinate frame includes: receiving images from an endoscopic camera; segmenting the images into a first dataset that includes image data of the surgical robotic tool and a second dataset that includes image data of tissue; passing the first and second datasets to a tool tracker and a tissue tracker, respectively; receiving pose information of the surgical robotic tool from the tool tracker and receiving the deformable 3D tissue reconstruction from the tissue tracker; combining the pose information and the deformable 3D tissue reconstruction into a common coordinate frame to provide information for generating a virtual surgical environment captured by the endoscopic camera.

In accordance with one particular implementation, combining the pose information and the deformable 3D tissue reconstruction further includes passing specified information between the tool tracker and the tissue tracker for improving the pose information and the deformable 3D tissue reconstruction, wherein the specified information includes surgical robotic tool manipulation data from the pose information and collision information from the deformable 3D tissue reconstruction.

In accordance with another particular implementation, the surgical robotic tool manipulation data includes tensioning, cautery and dissecting data.

In accordance with another particular implementation, segmenting the images further includes rendering of the surgical robotic tool to remove pixel information associated with the surgical robotic tool so that a remainder of the images includes the second dataset and excludes the pixel information associated with the tool.

In accordance with another particular implementation, the common coordinate frame is an endoscopic camera frame.

In accordance with another particular implementation, the tissue tracker performs tissue tracking and fusion.

In accordance with another particular implementation, the deformable 3D tissue reconstruction is a 3D surfel model.

This Summary is provided to introduce a selection of concepts in a simplified form. The concepts are further described in the Detailed Description section. Elements or steps other than those described in this Summary are possible, and no element or step is necessarily required. This Summary is not intended to identify key features or essential features of the claimed subject matter, nor is it intended for use as an aid in determining the scope of the claimed subject matter. The claimed subject matter is not limited to implementations that solve any or all disadvantages noted in any part of this disclosure.

### BRIEF DESCRIPTION OF THE DRAWINGS

FIG. 1 shows a simplified functional block diagram of one example of the various components and information sources for a system that performs surgical scene reconstruction, where solids lines show data flow requirements and dashed lines show optional informational input.

FIG. 2 shows one example of a surgical robotic tool illustrating its kinematics.

FIG. 3 shows point and edge features being detected on a surgical tool for estimating its location in 3D (left column of images) and a re-projection of that estimation (right column of images).

FIG. 4 illustrates the operation of one example of the synthesize tracking module shown in FIG. 1.

FIG. 5 is a flowchart illustrating one example of a method performed the surgical tool tracking module of FIG. 1, which tracks the Lumped Error and Observable Joint Angle Measurement Errors to generate pose information of the surgical robotic tool.

FIG. 6 is a flowchart illustrating one example of a method performed by the tissue tracking and fusing module of FIG. 1, which fully describes the tissue captured from endoscopic image in 3D using a surfel set and tracks the tissues deformations with Embedded Deform (ED) Nodes.

FIG. 7 is a flowchart illustrating one example of a method performed by synthesize tracking module of FIG. 1, which manages the endoscopic image(s) data stream, surgical tool tracking module, and the tissue tracking and fusion module.

### DETAILED DESCRIPTION

Described herein is a surgical perception framework or system, denoted SuPer, which integrates visual perception from endoscopic image data with a surgical robotic control loop to achieve tissue manipulation. A vision-based tracking system is used to track both the surgical environment and robotic agents. However, endoscopic procedures have limited sensory information provided by endoscopic images and take place in a constantly deforming environment. Therefore, we separate the tracking system into two methodologies: surgical tool tracking and tissue tracking and fusion. The two separate components are then synthesized together to perceive the entire surgical environment in 3D space. In some embodiments there may be one, two or more surgical tools in the environment and the surgical tool tracking module 25 is able track all them.

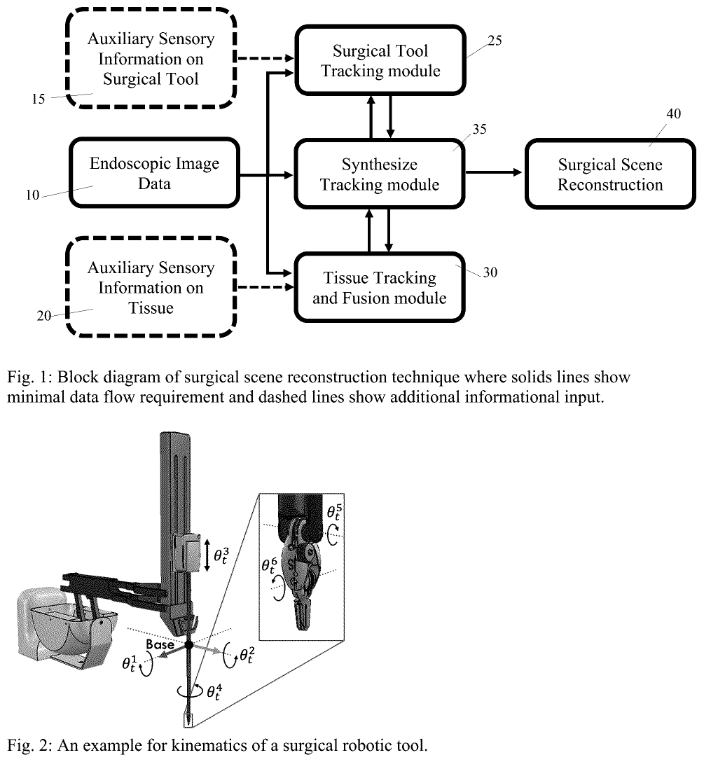

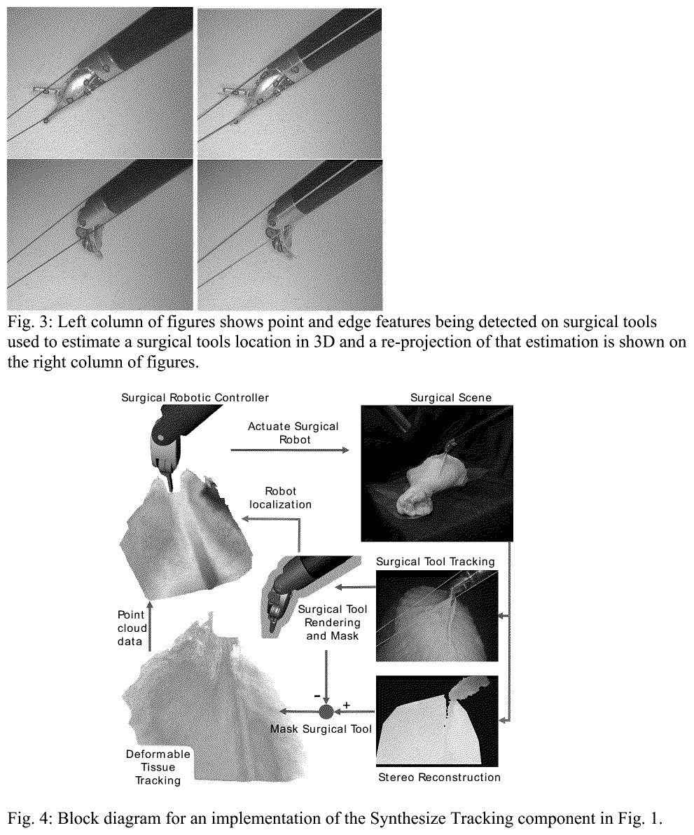

FIG. 1 shows a simplified functional block diagram of one example of the various components and information sources for a system that performs surgical scene reconstruction. As shown, the information that is used by the system includes endoscopic image data 10 (simply referred to herein as “images”) from one or more endoscopic cameras and, optionally, auxiliary sensory tissue information 20 and auxiliary sensor information 15 concerning the surgical tool or tools. Examples of auxiliary sensory information may include, without limitation, joint angle measurements from surgical tool encoders or the like, pre-operative CT/MRI scans, and ultra-sound.

The system also includes a surgical tool tracking component or module 25, a tissue tracking and fusion component or module 30 and a synthesize tracking component or module 35. The surgical tool tracking module 25 and the tissue tracking and fusion module 30 receive the endoscopic image data and the optional information, if available. The surgical tool tracking module 25 and the tissue tracking and fusion module 30 are also in communication with one another and with the synthesize tracking module 35, which also receives the endoscopic image data and provides as its output the reconstructed surgical scene 40.

The reconstructed surgical scene from the surgical perception framework or system described herein can be used by surgical robotic controllers to manipulate the surgical environment in a closed loop fashion as the framework maps the environment, tracking the tissue deformation and the surgical tools continuously and simultaneously. Furthermore, the SuPer framework also may be used in non-robotic automation applications (e.g. enhanced visualization for surgeons) and applied to any endoscopic surgical procedure, as the only required input is endoscopic image data. Illustrative embodiments of the various modules of the system will be described below. The first module that will be described performs surgical robotic tool tracking using a Bayesian filtering approach to understand the surgical robotic tools in 3D space. The second module that is discussed performs tissue tracking and fusion to track tissue deformations through a less dense graph of Embedded Deform (ED) nodes. Lastly, the synthesize tracking module 35 is discussed, which combines surgical tool tracking information and tissue tracking and fusion information into a single unified world that allows the surgical environment to be fully perceived in 3D.

### Surgical Robotic Tool Tracking

Surgical tool tracking provides a 3D understanding that shows where the surgical tool is located relative to the endoscopic camera or cameras. For illustrative purposes only the illustrative method will be limited to the tracking of a single surgical robotic tool from a single endoscopic camera. However, those of ordinary skill will recognize that these techniques may be extended to track multiple surgical robotic tools from multiple cameras. A challenge with surgical tool tracking is that endoscopes are designed to only capture a small working space for higher operational precision and hence only a small part of the surgical tool is typically visible. The method of tracking surgical robotic tools performed by the surgical tool tracking module 25 of FIG. 1 will be described for illustrative purposes only as using optional auxiliary sensor information from the robotic platform (e.g. joint angle measurements from an encoder). As noted above, however, in other applications (e.g. non-robotic) of the SuPer framework, alternative surgical tool tracking methods may be employed which do not use such auxiliary sensor information.

One example of a surgical robotic tool and its kinematics is shown in FIG. 2. Kinematics refer to the joints and links of the surgical robotic tool and hence fully describe it in 3D relative to its own base. Information concerning the links (i.e. the connecting parts between joints) are generally known from the robotic manufacturer and joint angle measurements are available from sensors such as encoders. Given the kinematics and a base to camera transform, the entire surgical robotic tool can be fully understood in 3D (e.g. pose information) with respect to the endoscopic camera. However, cable drives are typically utilized to actuate surgical robotic tools which enables low-profile robotic tools. These cable drives may cause joint angle measurement errors through stretch and other mechanical phenomena. Furthermore, the bases of the surgical robotic tools are adjusted regularly depending on the type of procedure and to fit each patient's anatomy. The surgical robotic tool tracking method described herein estimates these uncertainties and can be applied in real-time or for post processing of endoscopic images. It also generalizes to any joint angle measurement errors (e.g. backlash).

The 3D geometry of a surgical robotic tool can be fully described in the camera frame through a base-to-camera transform and forward kinematics. Details concerning the transformation matrices and robot kinematics may be found in B. Siciliano et. al, “Springer handbook of robotics,” vol. 200, Springer 2000. Mathematically the transform from the j-th link to the camera frame can be expressed as follows:

$${T}_{b}^{c} {\prod }_{i = 1}^{j} {T}_{i}^{i - 1} ( {\tilde{θ}}_{t}^{i} + {e}_{t}^{i} )$$

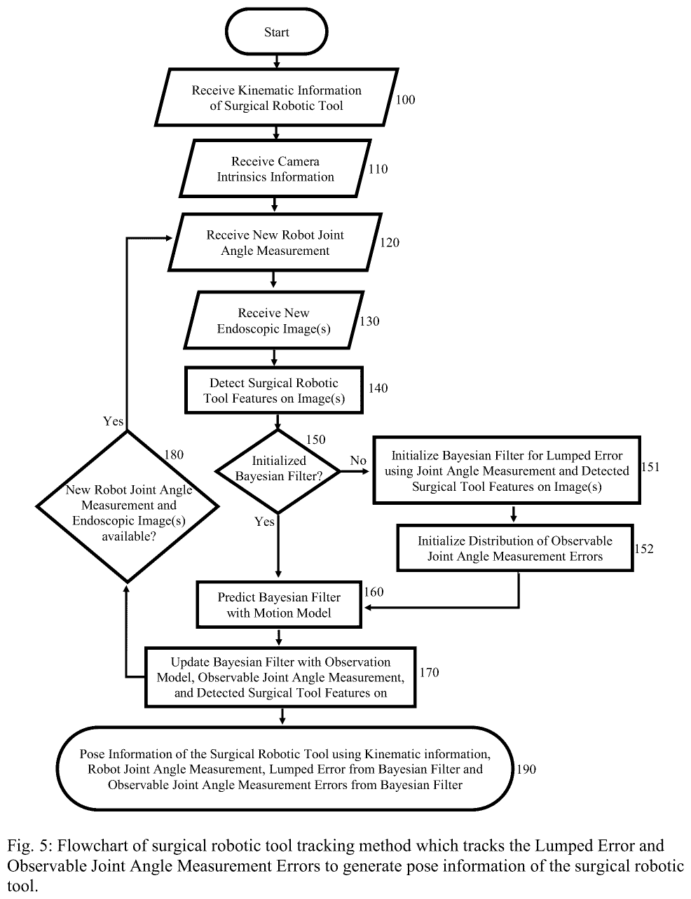

at time t where T_{b}^{c} ε SE(3) is the base-to-camera transform, Tᵢⁱ⁻¹(·) ε SE(3) is the i-th joint transform, \tilde{θ}ₜⁱ is the joint angle measurements, and eₜⁱ is the joint angle measurement error (i.e. θₜⁱ=\tilde{θ}ₜⁱ+eₜⁱ is the true joint angle). The joint transforms, Tᵢⁱ⁻¹(·), are provided by the surgical robotic tool manufacturer (see step 100 of FIG. 5). New joint angle measurements, \tilde{θ}ₜⁱ, and endoscopic images of the surgical robotic tool are received by the surgical tool tracking module 25 in steps 120 and 130 of FIG. 5, respectively. It has been demonstrated that solving for all the unknowns explicitly (T_{b}^{c} and eₜⁱ) is not possible when only a portion of the kinematic chain is visible in the camera frame (see F. Richter, J. Lu, R. K. Orosco and M. C. Yip, “Robotic Tool Tracking Under Partially Visible Kinematic Chain: A Unified Approach,” in IEEE Transactions on Robotics, doi: 10.1109/TRO.2021.3111441). Therefore, we collect the terms that cannot be estimated into a single Lumped Error transform:

$${T}_{{n}_{b}}^{c} {\prod }_{i = 1}^{{n}_{b}} {T}_{i}^{i - 1} ( {\tilde{θ}}_{t}^{i} ) {\prod }_{i = {n}_{b} + 1}^{j} {T}_{i}^{i - 1} ( {\tilde{θ}}_{t}^{i} + {e}_{t}^{i} )$$

where Tₙ_{_{b}}^{c} ε SE(3) is the Lumped Error and all the kinematic links preceding joint n_{b} are out of the camera frame. Intuitively, the Lumped Error transform is virtually adjusting the base of the kinematic chain for the robot in the camera frame. The virtual adjustments are done to fit the error of the first n_{b} joint angles and the base-to-camera transform. The Lumped Error transform and the observable joint angle measurement errors eₜⁿ^{_{b}}, eₜⁿ^{_{b}}⁺¹, . . . also can be estimated while fully describing all the visible links from the surgical robotic tool in the camera frame. Furthermore, it is a significant reduction of the parameters that need to be estimated for surgical robotic tool tracking than the original problem.

A Bayesian Filtering technique may be used to track the unknown parameters that need to be estimated, Tₙ_{_{b}}^{c} and eₜⁿ^{_{b}}, eₜⁿ^{_{b}}⁺¹, . . . . The Bayesian Filter requires motion and observation models to be defined. Once these are defined, any Bayesian Filtering technique can be used to solve for the unknown parameters (e.g. Kalman Filter and Particle Filter). Details concerning Bayesian Filtering techniques and Kalman filters may be found in Z. Chen, “Bayesian filtering: from Kalman filters to particle filters and beyond,” in Statistics, vol. 182, no. 1, pp. 1-69, 2003.

In the coming two sub-sections, motion and observation models are defined to estimate Tₙ_{_{b}}^{c} and eₜⁿ^{_{b}}, eₜⁿ^{_{b}}⁺¹, . . . , with a Bayesian Filter. By estimating these parameters, the surgical robotic tool can be described in 3D (e.g. its pose) with respect to the endoscopic camera frame (see step 190 in FIG. 5). The information describing a surgical robotic tool in 3D can be used for a multitude of applications such as closed loop control and enhanced visualization for surgeons, for example.

Motion Model: The Lumped Error, Tₙ_{_{b}}^{c}, is estimated with an axis angle vector, ŵₜ, and translation vector, \hat{b}ₜ. Their initial values (i.e. ŵ₀, \hat{b}₀) are set to an initial, coarse calibration of the base-to-camera transform, T_{b}^{c} (e.g. SolvePnP from point features) (see step 151 in FIG. 5). Then, the motion model is defined as follows:

[ŵₜ,\hat{b}_{c}]^{T}˜ [symbol] ([ŵₜ₋₁,\hat{b}ₜ₋₁]^{T},Σ_{w,b,t})

where Σ_{w,b,t} is the covariance matrix. A Weiner Process is once again chosen for the same reason as the joint angle measurement error motion model (see step 160 in FIG. 5)

The vector of observable joint angle measurement errors being estimated, êₜ, are initialized from a uniform distribution and have a motion model of additive zero mean Gaussian noise:

ê₀˜ [symbol] (−aₑ,aₑ)

êₜ˜ [symbol] (êₜ₋₁,Σ_{e,t})

where aₑ describes the bounds of constant joint angle measurement error and Σ_{e,t} is the covariance matrix. The initialization is done to capture joint angle biases, and a Weiner Process is chosen for the motion model due to its ability to generalize over a large number of random processes. The initialization and motion models of the joint angle measurement errors are performed in steps 152 and 170 of FIG. 5, respectively.

Observation Model: To update the parameters being estimated, ŵₜ, \hat{b}ₜ, êₜ, from endoscopic images, features need to be detected and a corresponding observation model for them must be defined. The coming observation models will generalize for any point or edge features. Examples of these detections are shown in FIG. 3. In FIG. 3, colored markers (shown in grayscale) are used to detect point features and the edges of the surgical robotic tool's insertion shaft are used as edge features of a cylindrical shape. These point and edge features can be detected via fiducial markers, classical features (e.g. canny-edge detector), or deep learned features. The feature detection of surgical robotic tool from endoscopic images is performed in steps 140 of FIG. 5. The remainder of this sub-section defines observation models for these detected features to update the parameters being estimated, ŵₜ, \hat{b}ₜ,êₜ.

Let mₜ be a list of detected point features in the image frame from the surgical robotic tool. By following the standard camera pin-hole model, the camera projection equation for the k-th point at position p^{jk} on joint jₖ is:

$${\hat{m}}_{t} = \frac{1}{s} KT ( {\hat{w}}_{t} , {\hat{b}}_{t} ) {\prod }_{i = 1}^{{n}_{b}} {T}_{i}^{i - 1} ( {\tilde{θ}}_{t}^{i} ) {\prod }_{i = {n}_{b} + 1}^{{j}_{k}} {T}_{i}^{i - 1} ( {\tilde{θ}}_{t}^{i} + {e}_{t}^{i} ) {\overline{p}}^{{j}_{k}}$$

where

$$\frac{1}{s} K$$

is the camera projection operator with intrinsic matrix K and T(ŵₜ, \hat{b}ₜ) ε SE(3) is the homogeneous representation of ŵₜ, \hat{b}ₜ, and τ is homogeneous representation of a point (e.g. p̅=[p, 1]^{T}). In step 110 of FIG. 5, the camera intrinsics, K, are received by the surgical robotic tool tracking module and can be estimated using camera calibration techniques which are known by those of ordinary skill.

Similarly, let the paired lists ρₜ, ϕₜ be the parameters describing the detected edges in the image from the surgical robotic tool. The parameters describe an edge in the image frame using the Hough Transform, so the k-th pair, ρₜ^{k}, ϕₜ^{k}, parameterize the k-th detected edge with the following equation:

ρₜ^{k}=u cos(ϕₜ^{k})+v sin(ϕₜ^{k})

where (u, v) are pixel coordinates. Using the estimates, ŵₜ, \hat{b}ₜ, êₜ, let the k-th edge be defined as \hat{ρ}ₜ^{k}, \hat{ϕ}ₜ^{k} after projecting the k-th edge onto the image plane. These projection equations will need to be defined based on the geometry of the surgical robotic tool. An example of a cylindrical shape and others are derived in B. Espiau, et. al., “A new approach to visual servoing in robotics” Transactions on Robotics and Automation, vol. 8, no. 3, pp. 313-326, 1992.

From the point and edge detections and corresponding projection equations, probability distributions can be defined for observation models for the Bayesian Filters. For the list of point features, the probability is:

$$P ( {m}_{t} \vert {\hat{w}}_{t} , {\hat{b}}_{t} , {\hat{e}}_{t} ) = {\sum }_{k} {e}^{- {γ}_{m} ‖ {m}_{t}^{k} - {\hat{m}}_{t}^{k} ‖}$$

where γₘ is a tuned parameter and adjusts the confidence of point feature detections. Similarly, the probability of the list of detected edges is:

$$P ( {ρ}_{t} , {ϕ}_{t} \vert {\hat{w}}_{t} , {\hat{b}}_{t} , {\hat{e}}_{t} ) = {\sum }_{k} {e}^{- {γ}_{ρ} \vert {ρ}_{t}^{k} - {\hat{ρ}}_{t}^{k} \vert - {γ}_{ϕ} \vert {ϕ}_{t}^{k} - {\hat{ϕ}}_{t}^{k} \vert}$$

where γ_{ρ} and γ_{ϕ} is a tuned parameter and adjusts the confidence of edge feature detections. The probability distributions can be viewed as a summation of Gaussians centered about the projected features where the standard deviations are adjusted via γₘ, γ_{ρ}, γ_{ϕ}. The observation models are employed in step 170 of FIG. 5 to update the estimation of ŵₜ, \hat{b}ₜ, êₜ in the Bayesian Filter.

### Tissue Tracking and Fusion

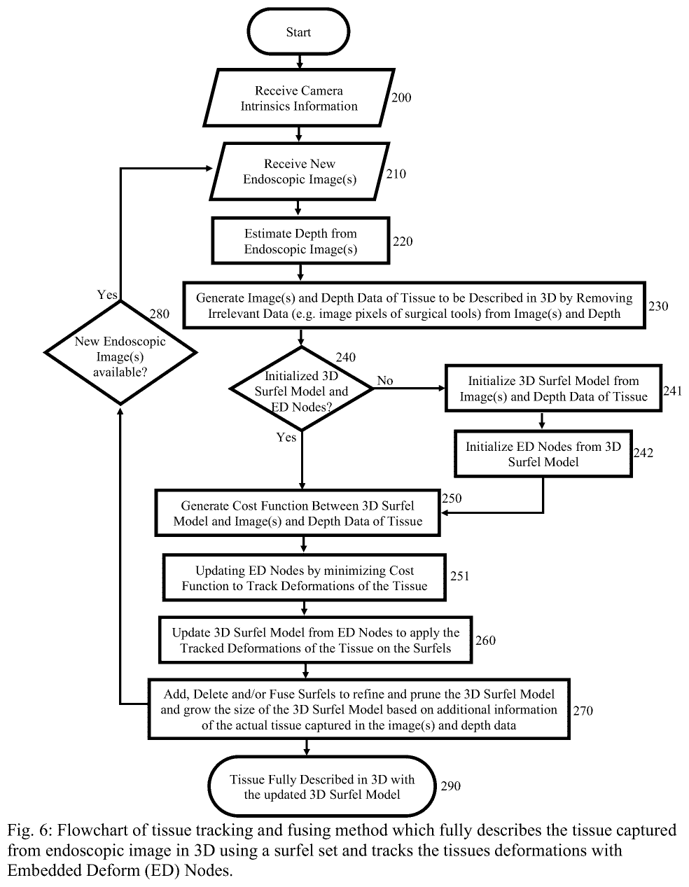

The tissue tracking and fusion module 30 shown in FIG. 1 takes in endoscopic image data and outputs a deformable 3D reconstruction of the actual tissue in the surgical site. This section described one particular embodiment of a surgical tissue tracking technique where a less dense graph of ED nodes is used to track the deformations of the tissue while simultaneously fusing multiple endoscopic image(s) to create a panoramic scene of the tissue. FIG. 6 is a flowchart illustrating this particular method. As input, the method takes in endoscopic image(s) of the surgical scene, as shown in step 210 of FIG. 6. Depth is generated from the image(s), as shown in step 220, which can be accomplished using stereo-endoscopes with pixel matching or using mono-endoscopes and directly estimating depth (using e.g., deep learning techniques). If there are other objects are in the image(s) and depth data (e.g. surgical tools or even tissue not of interest), that data must be removed in step 230. Approaches for removing non-tissue related image data are described below in the section discussing the synthesize tracking module 35.

To represent the tissue, we choose surfel as our data structure due to the direct conversion to point cloud, which is a standard data type for the robotics community. A surfel S represents a region of an observed surface as a disk and is parameterized by the tuple (p, n, c, r) where p, n, c, r are the expected position, normal, color, and radius respectively. A 3D surfel model is initialized from the first image(s) and depth data, as described in Keller et. al “Surfelwarp: Efficient non-volumetric single view dynamic reconstruction,” RSS, 2018. The surfel initialization is performed in step 241 of FIG. 6.

Since the number of surfels grows proportionally to the number of image pixels provided to the tissue tracking and fusion module 30, it is infeasible to track the entire surfel set individually. We drive our surfel set with a less-dense ED graph. With a uniform sampling from the surfel to initialize the ED nodes, the number of ED nodes is much fewer than the number of surfels. Thus, the ED graph has significantly fewer parameters to track compared with the entire surfel model. The initialization of the ED nodes is performed in step 242 of in FIG. 6. Moreover, the ED graph can be thought of as an embedded sub-graph and skeletonization of the surfels to capture their deformations. The transformation of every surfel position is modeled as follows:

$${\overline{p}}^{′} = {T}_{g} {\sum }_{i ∈ KNN ( p )} {α}_{i} ( {T}_{i} ( \overline{p} - {\vec{g}}_{i} ) + {\vec{g}}_{i} )$$

where T_{g} ε SE(3) is the common motion shared across all surfels (e.g. camera motion), KNN(p) is the set of ED nodes indices which are the k-th nearest neighbors of p, αᵢ is a normalized weight (as computed in R. W. Sumner et. al “Embedded deformation for shape manipulation”, Transactions on Graphics, vol. 26, no. 3, pp 80-es, ACM, 2007), Tᵢ ε SE(3) is the local transformation of the i-th ED node, gᵢ is the position of the i-th ED node, and \vec{·} is homogeneous representation of a vector (e.g. \vec{g}=[g, 0]^{T}). The normal transformation is similarly defined as:

$${\vec{n}}^{′} = {T}_{g} {\sum }_{i ∈ KNN ( p )} {α}_{i} {T}_{i} \vec{n}$$

To track the visual scene with the parameterized surfels, a cost function is defined to represent the loss between image(s) and depth data of the tissue and the 3D surfel model. It is defined as follows:

E_{data}+λₐEₐᵣₐₚ+λᵣEᵣₒₜ+λ_{c}E_{corr}

where E_{data} is error between the depth observation and estimated model (e.g. normal-difference cost), Eₐᵣₐₚ is a rigidness cost such that ED nodes nearby one another have similar deformation (e.g. as-rigid-as-possible cost), Eᵣₒₜ is a normalization term to ensure the rotational components of Tᵢ and T_{g} lie on the SO(3) manifold, and E_{corr} is a visual feature correspondence cost to ensure texture consistency. Mathematical details concerning the specific costs may be found in Y. Li et. al., “Super: A surgical perception framework for endoscopic tissue manipulation with surgical robotics” RA-L, vol. 5, no. 2, pp. 2294-2301, IEEE, 2020. Note that some of the cost terms will require camera intrinsics (see step 100 of FIG. 6). The generation of the cost function is accomplished in step 150 of FIG. 6.

The cost function between the 3D surfel model and the image(s) and depth data of the tissue is minimized to solve for the ED nodes local transformations, Tᵢ, which represent the deformations of the tissue. Step 251 of FIG. 6 solves for the ED nodes. After every frame, the deformations are committed to each surfel's position and normal (e.g. p′→p and n′→n). The surfels are updated in step 260 of FIG. 6. Lastly, the 3D surfel model itself is modified by adding, deleting, and/or fusing of surfels, as done in Keller et. al “Surfelwarp: Efficient non-volumetric single view dynamic reconstruction,” RSS, 2018. The adding/deleting and fusing of surfels is performed in step 270 of FIG. 6. This step is used to refine and prune the 3D surfel model and grow the size of the 3D surfel model as new information of the tissue is captured from the image(s) and depth data.

The updated 3D surfel model fully describes the tissue of interest in 3D with respect to the endoscopic camera. This output is shown in step 290 of FIG. 6. Furthermore, it is fully described over time because the ED nodes track the deformations of the tissue. This can be applied to downstream surgical applications such as closed loop control for surgical robotics, where locations on the tissue are kept track of even as the tissue deforms. The surfel set can also be used to enhance visualization for surgeons during an endoscopic surgery.

### Synthesize Tracker

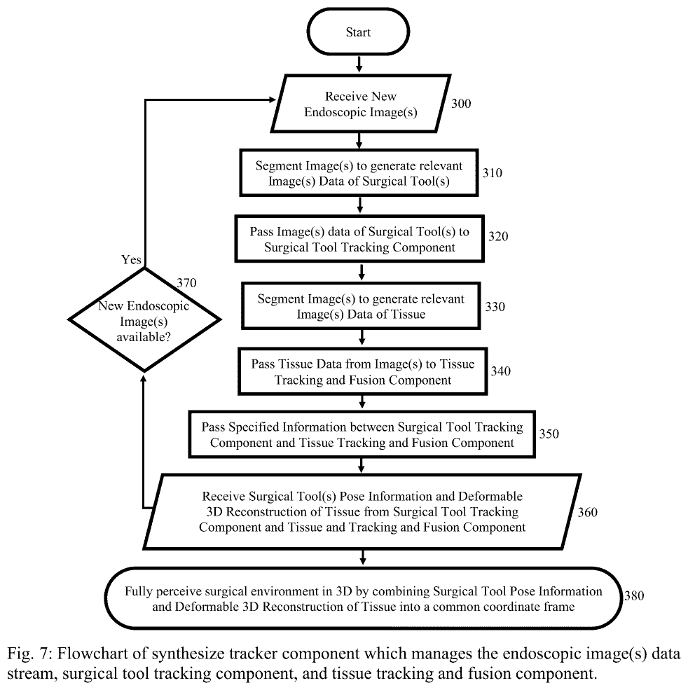

The synthesize tracking module 35 interfaces between the surgical tool tracking module 25 and tissue tracking and fusion module 35 shown in the framework of FIG. 1. A flowchart illustrating one example of the method performed by this module is shown in in FIG. 7. The output from the synthesize tracking module 35 is the information necessary for generating a virtual surgical environment, which is generated using the endoscopic image data as input, passing the appropriate image(s) data to the appropriate module, and finally combining the outputs of the surgical tool tracking module 25 and tissue tracking and fusion module 30 into a common coordinate frame. The input of endoscopic image(s) is received by the synthesize tracking module 35 in step 300 of FIG. 7.

In order to pass the necessary endoscopic image(s) data to the appropriate modules, the image(s) are segmented in steps 310 and 330, respectively, to generate image(s) data of surgical tool and image(s) data of tissue. An example of this process is shown in FIG. 4, where the surgical tool tracking module 25 takes in the entire endoscopic image(s) (i.e. no segmentation necessary) and the image(s) data of tissue is generated by masking out pixels of the endoscopic image(s) data from a rendered mask of the surgical tool. Alternative way to perform the segmentation include deep learning techniques that segment the image(s) to find the pixels associated with the surgical tools and tissue. The segmented data is passed to the surgical tool tracking module 25 and the tissue tracking and fusion module 30 in steps 320 and 340, respectively. With reference to the previous section concerning surgical robotic tool tracking, no segmentation was required as the feature detection algorithm, which is used in step 140 of FIG. 5, can operate on the entire endoscopic image(s) data. Meanwhile tissue data is segmented in step 230 of FIG. 6, as described in the previous section concerning tissue tracking and fusion.

Once the appropriate endoscopic image(s) data is passed to the two modules to perform surgical tool tracking and tissue tracking and fusion, specified information is shared between them to improve the outputs from each of them. This sharing of information is shown in step 350 of FIG. 7. An example of the type of specified information that may be shared is manipulation information (e.g. tensioning, cautery, dissecting) available from the surgical tool tracking module 25. In the instance of dissection, the information specifying where a dissection occurs on a tissue can be leveraged by the tissue tracking and fusion module 30 to update its deformable 3D reconstruction model regarding the location of the tissue dissection. In the specific instance of 3D surfel modelling as presented in the previous section concerning tissue tracking and fusion, the ED nodes will not deform surfels across the location of a dissection, hence keeping the deformations on either side of a dissection independent of one another. Likewise, the tissue tracking and fusion module provides collision information concerning locations where the surgical tool cannot be found (e.g. inside the tissue). The collision information can be applied as a constraint to the tracked surgical tool and standard iterative, collision solvers can be applied to push the tracked surgical tools out of collision with the tissue.

The outputs from the surgical tool tracking module 25 and tissue tracking and fusion module 30 are collected and combined to fully perceive the surgical site in 3D (see step 160 in FIG. 7). The surgical tool tracking module 25 provides pose information of the surgical tools and the tissue tracking and fusion component provides a deformable 3D reconstruction of the actual tissue. By combining the two outputs into a unified world, downstream surgical applications can utilize the fully perceived surgical site in 3D. For example, closed loop control in cases of surgical robotic tools and enhanced visualization for surgeons.

### CONCLUSION

Several aspects of the SuPer framework are presented in the foregoing description and illustrated in the accompanying drawing by various blocks, modules, components, steps, processes, algorithms, etc. (collectively referred to as “elements”). These elements may be implemented using electronic hardware, computer software, or any combination thereof Whether such elements are implemented as hardware or software depends upon the particular application and design constraints imposed on the overall system. By way of example, an element, or any portion of an element, or any combination of elements may be implemented with a “processing system” that includes one or more processors. Examples of processors include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionalities described throughout this disclosure

Various embodiments described herein may be described in the general context of method steps or processes, which may be implemented in one embodiment by a computer program product, embodied in, e.g., a non-transitory computer-readable memory, including computer-executable instructions, such as program code, executed by computers in networked environments. A computer-readable memory may include removable and non-removable storage devices including, but not limited to, Read Only Memory (ROM), Random Access Memory (RAM), compact discs (CDs), digital versatile discs (DVD), etc. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

A computer program product can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

The various embodiments described herein may be implemented in various environments. Such environments and related applications may be specially constructed for performing the various processes and operations according to the disclosed embodiments or they may include a general-purpose computer or computing platform selectively activated or reconfigured by code to provide the necessary functionality. Embodiments described herein may be practiced with various computer system configurations including hand-held devices, tablets, microprocessor systems, microprocessor-based or programmable consumer electronics, minicomputers, mainframe computers and the like. However, the processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the disclosed embodiments, or it may be more convenient to construct a specialized apparatus or system to perform the required methods and techniques. In some cases the environments in which various embodiments described herein are implemented may employ machine-learning and/or artificial intelligence techniques to perform the required methods and techniques.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

1. A method for tracking a surgical robotic tool being viewed by an endoscopic camera, comprising:
receiving images of the surgical robotic tool from the endoscopic camera;
receiving surgical robotic tool joint angle measurements from the surgical robotic tool;
detecting predetermined features of the surgical robotic tool on the images of the surgical robotic tool to define an observation model to be employed by a Bayesian Filter;
estimating a lumped error transform and observable joint angle measurement errors using the Bayesian Filter, the lumped error transform compensating for errors in a base-to-camera transform and non-observable joint angle measurement errors;
determining pose information over time of the robotic tool with respect to the endoscopic camera using kinematic information of the surgical robotic tool, the surgical robotic tool joint angle measurements, the lumped error transform estimated by the Bayesian Filter and the observable joint angle measurement errors estimated by the Bayesian Filter; and
providing the pose information to a surgical application for use therein.

2. The method of claim 1 wherein the surgical application is a closed loop control system for controlling the robotic tool in a frame of view of the endoscopic camera.

3. The method of claim 1 wherein the surgical application is configured to render the surgical robotic tool using the pose information.

4. The method of claim 3 wherein the surgical robotic tool is rendered for use in an artificial reality or virtual reality system.

5. The method of claim 1 wherein the surgical robotic tool and the endoscopic camera are located at a surgical site.

6. The method of claim 1 wherein the endoscopic camera is incorporated in an endoscope incorporated in a robotic system that includes the surgical robotic tool.

7. The method of claim 1 wherein the endoscopic camera is incorporated in an endoscope that is independent of a robotic system that includes the surgical robotic tool.

8. The method of claim 1 wherein the surgical robotic tool joint angle measurements are received from encoders associated with the surgical robotic tool.

9. The method of claim 1 wherein detecting predetermined features of the surgical robotic tool includes detecting point features.

10. The method of claim 9 wherein detecting the point features is performed using a deep learning technique or fiducial markers.

11. The method of claim 1 wherein the predetermined features are edge features.

12. The method of claim 11 wherein detecting the edge features is performed using a deep learning algorithm or a canny edge detection operator.

13. A method for tracking tissue being viewed by an endoscopic camera, comprising:
receiving images of the tissue from the endoscopic camera;
estimating depth from the endoscopic images;
initializing a three-dimensional (3D) model of the tissue with surfels from an initial one of the images and the depth data of the tissue to provide a 3D surfel model;
initializing embedded deformation (ED) nodes from the surfels, wherein the ED nodes apply deformations to the surfels to mirror actual tissue deformation;
generating a cost function representing a loss between the images from the endoscopic camera and the depth data of the tissue and the 3D surfel model;
updating the ED Nodes by minimizing the cost function to track deformations of the tissue;
updating the surfels from the ED nodes to apply the tracked deformations of the tissue on the surfels; and
adding surfels to grow a size of the 3D Surfel model based on additional information of the actual tissue that is subsequently captured in the images and the depth data to provide an updated 3D surfel model for use in a surgical application.

14. The method of claim 13 wherein adding surfels further comprises adding, deleting and/or fusing the surfels to refine and prune the 3D surfel model and grow a size of the 3D surfel model based on additional information of the actual tissue that is subsequently captured in the images and the depth data.

15. The method of claim 13 wherein the cost function is minimized by an optimization technique selected from the group including gradient descent, a Levenberg Marquardt algorithm and coordinate descent.

16. The method of claim 13 wherein estimating depth from endoscopic images is performed using a stereo-endoscope and pixel matching or by directly estimating depth from a mono endoscope using a deep learning technique.

17. The method of claim 13 further comprising removing irrelevant data from the images and the depth data.

18. The method of claim 17 wherein the irrelevant data includes image pixels of a surgical tool.

19. The method of claim 13 wherein the cost function includes a normal-difference cost.

20. The method of claim 13 wherein the cost function includes a rigid-as-possible cost.

21. The method of claim 13 wherein the cost function includes a rotational normalizing cost to constrain a rotational component of the ED nodes to the rotational manifolds.

22. The method of claim 13 wherein the cost function includes a texture loss between matched feature points though matched feature point pairs.

23. The method of claim 13 wherein the surgical application is a closed loop control system for controlling a robotic tool in a frame of view of the endoscopic camera.

24. The method of claim 13 wherein the surgical application is configured to render the tissue using the updated 3D surfel model.

25. A method for synthesizing surgical robotic tool pose information and a deformable 3D reconstruction of tissue into a common coordinate frame, comprising:
receiving images from an endoscopic camera;
segmenting the images into a first dataset that includes image data of the surgical robotic tool and a second dataset that includes image data of tissue;
passing the first and second datasets to a tool tracker and a tissue tracker, respectively;
receiving pose information of the surgical robotic tool from the tool tracker and receiving the deformable 3D tissue reconstruction from the tissue tracker;
combining the pose information and the deformable 3D tissue reconstruction into a common coordinate frame to provide information for generating a virtual surgical environment captured by the endoscopic camera.

26. The method of claim 25 wherein combining the pose information and the deformable 3D tissue reconstruction further includes passing specified information between the tool tracker and the tissue tracker for improving the pose information and the deformable 3D tissue reconstruction, wherein the specified information includes surgical robotic tool manipulation data from the pose information and collision information from the deformable 3D tissue reconstruction.

27. The method of claim 26 wherein the surgical robotic tool manipulation data includes tensioning, cautery and dissecting data.

28. The method of claim 25 wherein segmenting the images further includes rendering of the surgical robotic tool to remove pixel information associated with the surgical robotic tool so that a remainder of the images includes the second dataset and excludes the pixel information associated with the tool.

29. The method of claim 25 wherein the common coordinate frame is an endoscopic camera frame.

30. The method of claim 25 wherein the tissue tracker performs tissue tracking and fusion.

31. The method of claim 25 wherein the deformable 3D tissue reconstruction is a 3D surfel model.
